(19) United States
(12) Patent Application Publication
(10) Pub. No.: US 2025/0000529 A1
(43) Pub. Date: Jan. 2, 2025

(54) FLOW CONTROL SWITCH WITH VARIABLE FLOW RATE CONTROL AND WITH CONSISTENT CLOG-FREE INNER DIAMETER

(71) Applicant: KONINKLIJKE PHILIPS N.V., EINDHOVEN (NL)

(72) Inventor: Richard Charles GUNDERSON, SAINT PAUL, MN (US)

(21) Appl. No.: 18/696,417

(22) PCT Filed: Sep. 13, 2022
(86) PCT No.: PCT/EP2022/075365

§ 371 Date: Mar. 28, 2024

Related U.S. Application Data
(60) Provisional application No. 63/251,817, filed on Oct. 4, 2021.

(51) Int. Cl. A61B 17/22

(52) U.S. Cl. CPC A61B 17/22; A61B 2017/22079; A61B 2217/005

(57) ABSTRACT

A thrombectomy device (10) includes a hemostasis valve Y connector (12) including a distal end (14) configured to rotatably connect with an associated aspiration catheter (2); and a Y connector (16) connecting the distal end with a first branch (18) terminating in a hemostasis valve (20) and a second branch (22) configured to connect with an associated vacuum tube (4) of an associated aspiration pump (8). An adjustable valve (26) is integrated into the hemostasis valve Y connector or configured to connect in-line with the associated vacuum tube. The adjustable valve is configured to adjust a flow rate of fluid drawn by the associated aspiration pump and having a closed position with zero flow rate, a maximum open position with a maximum flow rate, and at least one intermediate position with a flow rate intermediate between zero flow rate and the maximum flow rate.

### FIELD

The following relates generally to the catheter arts, aspiration catheter arts, thrombectomy arts, and related arts.

### BACKGROUND

Vascular therapy (e.g., thrombectomy, atherectomy, and so forth) devices are medical devices designed to remove or modify tissue or material from inside a diseased vessel (e.g., an artery, a vein, etc.). In particular, mechanical thrombectomy by direct aspiration is an effective treatment for vascular clots. In such procedures, the tip of an aspiration catheter is deployed at the site of the clot, and suction is applied to draw out the clot. The applied vacuum is controlled to preferentially remove the clot material while minimizing blood loss.

An aspiration catheter can typically include a clamp (such as a roller clamp or a pinch clamp) to control a flow of liquids, solids, and gases through the catheter under applied suction. However, such clamps lack precision for flow control as the clamp compresses or squishes the suction tube. In addition, an unlocking and locking of the clamp can be difficult and cumbersome, prolonging a delay in flow control.

In addition, thrombectomy procedures can include mechanical aspiration. Such procedures employ a vacuum pump and a fluid collection system, with the suction tube connecting the fluid collection system to the catheter. However, such systems include a flow control switch that has a silicone tube therewithin that is pinched to close the tube. The switch has many ledges and diameter changes within it, which can be catch points for an aspirated clot and cause blockage of the suction tube.

The following discloses certain improvements to overcome these problems and others.

### SUMMARY

In some embodiments disclosed herein, a thrombectomy device includes a hemostasis valve Y connector including a distal end configured to rotatably connect with an associated aspiration catheter; and a Y connector connecting the distal end with a first branch terminating in a hemostasis valve and a second branch configured to connect with an associated vacuum tube of an associated aspiration pump. An adjustable valve is integrated into the hemostasis valve Y connector or configured to connect in-line with the associated vacuum tube. The adjustable valve is configured to adjust a flow rate of fluid drawn by the associated aspiration pump and having a closed position with zero flow rate, a maximum open position with a maximum flow rate, and at least one intermediate position with a flow rate intermediate between zero flow rate and the maximum flow rate.

In some embodiments disclosed herein, a thrombectomy device includes a hemostasis valve Y connector including a distal end configured to rotatably connect with an associated aspiration catheter; and a Y connector connecting the distal end with a first branch (terminating in a hemostasis valve and a second branch configured to connect with an associated vacuum tube of an associated aspiration pump. An adjustable valve is integrated into the hemostasis valve Y connector or configured to connect in-line with the associated vacuum tube. The adjustable valve includes a valve lumen; and a control mechanism configured to adjustably occlude the valve lumen to adjust the flow rate of fluid drawn by the associated aspiration pump. The adjustable valve is configured to adjust a flow rate of fluid drawn by the associated aspiration pump and having a closed position with zero flow rate, a maximum open position with a maximum flow rate, and at least one intermediate position with a flow rate intermediate between zero flow rate and the maximum flow rate.

In some embodiments disclosed herein, a thrombectomy method includes: positioning a catheter adjacent a treatment site of a patient; operating a hemostasis valve Y connector connected to the catheter to move a tip of the catheter radially around a blood vessel at the treatment site; operating an adjustable valve integrated into the hemostasis valve Y connector or configured to connect in-line with an associated vacuum tube connecting an aspiration pump with the hemostasis valve Y connector to initiate aspiration of material via the catheter; and adjusting the adjustable valve to adjust the aspiration to a low but not zero flow rate of aspirated material when aspirated material transitions from clot material to blood.

One advantage resides in providing an adjustable valve connected in-line on a suction tube or included in a hemostasis valve Y connector of a thrombectomy device configured to couple with an aspiration catheter.

Another advantage resides in providing an adjustable valve on a suction tube or hemostasis valve Y connector of such a thrombectomy device allowing precise control of flow through the suction tube.

Another advantage resides in providing an adjustable valve on a suction tube or hemostasis valve Y connector of a thrombectomy device that can be easily locked and unlocked.

Another advantage resides in providing such an adjustable valve in which the adjustable valve has a consistent inner diameter.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a thrombectomy apparatus in accordance with the present disclosure.

FIGS. 2-4 show components of the apparatus of FIG. 1.

FIGS. 5-7 show another embodiment of the thrombectomy device of FIG. 1.

FIG. 8 shows another embodiment of the thrombectomy device of FIG. 1.

FIGS. 9-12 show another embodiment of the thrombectomy device of FIG. 1.

FIG. 13 diagrammatically illustrates a method of performing a thrombectomy method using the apparatus of FIG. 1.

### DETAILED DESCRIPTION

A typical thrombectomy device or assembly for use in conjunction with an aspiration catheter employs a small battery powered pump to apply suction to a clot located at the tip of the aspiration catheter inserted into vasculature so as to suction clot material into a disposal container. The aspiration catheter includes a hemostasis valve Y connector with a proximal (patient-facing) end with a catheter rotation knob, an opposite distal end with a hemostatic valve cinched around the catheter to seal that end off, and a Y connector via which the pump couples to the catheter lumen inside the hemostasis valve Y connector.

During a thrombectomy procedure, precise control of the applied vacuum is desired to maximize suction removal of the clot while limiting the suctioned blood to, at most, a few hundred milliliters (mLs), and more preferably much less than that. In a typical commercial aspiration catheter, the operator has two suction controls: an on/off switch on the pump, and an in line pinch valve on the tube leading between the hemostasis valve Y connector and the pump. Two-handed operation is generally required, with one hand used to push the catheter in to engage the clot and to also operate the catheter rotation knob to sweep the catheter tip radially around the blood vessel lumen, and the other hand being used to operate the in-line pinch valve. The operator may open the pinch valve to suction clot material while rotating the catheter tip until blood starts flowing into the disposal container, then close the pinch valve to stop suction, push the catheter further in, and open the pinch valve to observe whether it is now suctioning clot material or blood, and repeat this sequence until the thrombectomy is complete. Fluoroscopy imaging guidance may be used, but the operator typically relies heavily on observation of the pumped material to assess whether the aspiration is removing clot material or blood at any given time.

In some embodiments disclosed herein, the in-line pinch valve is replaced by a continuous or multi-step adjustable valve. In general, the adjustable valve assembly can include a valve lumen and a push-type or rotary control that provides variable occlusion of the valve lumen. The adjustable valve assembly may provide continuous adjustment, or may provide multiple flow settings, e.g., by way of a rotary disk with multiple openings of different sizes that can be selectively aligned with the valve lumen by rotating a rotary control. The adjustable valve assembly may further include O-rings for sealing, and is preferably designed to have a uniform inner diameter to avoid blockage build-up inside the adjustable valve assembly.

In some embodiments, the adjustable valve assembly may be placed in-line on the tube extending between the Y adaptor and the pump. In this case, the valve assembly includes inlet and outlet tube couplings for connecting in-line, and the valve lumen extends between those tube couplings. In this design, the adjustable valve assembly is a direct replacement for the conventional pinch valve, but with improved functionality.

In other embodiments, the adjustable valve assembly may be integrated into the hemostasis valve Y connector, preferably located at or near the proximal (patient-facing) end along with the catheter rotation knob. This placement can enable one-hand operation, as the operator can use one hand to operate both the catheter rotation knob and the rotary knob of the adjustable valve assembly, while also pushing in the catheter as appropriate.

One-handed operation of the improved aspiration catheter can be as follows. A single hand can push the catheter in to engage the clot and to also operate the catheter rotation knob to sweep the catheter tip radially around the blood vessel lumen. That same hand can also open the adjustable valve assembly to initiate suction, until the suctioned clot material begins transitioning to suctioned blood. At that point, the single hand can be used to adjust the suction using the push-type or rotary control of the adjustable valve assembly while moving (and possibly rotating) the catheter to search for more clot material, observing the suctioned material (which is flowing at a slow rate now due to the throttling) to detect when the catheter tip engages clot material. By having a reduced, but nonzero, amount of suction applied while repositioning the catheter tip, the operator has positive feedback as to whether clot material or blood is being suctioned, while reducing blood loss by applying the reduced suction.

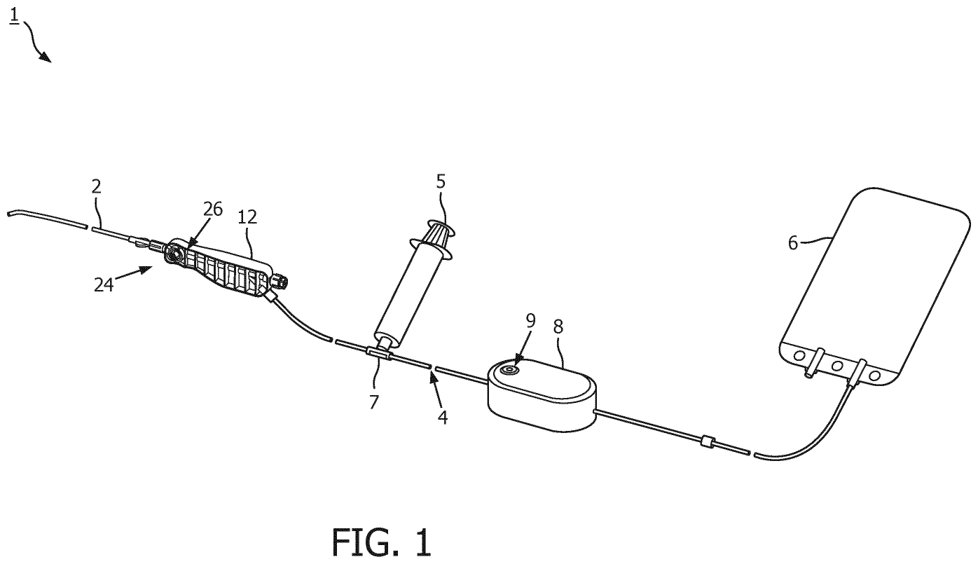

With reference to FIG. 1, an illustrative vascular therapy (i.e., thrombectomy or atherectomy) apparatus 1 is diagrammatically shown. As shown in FIG. 1, the apparatus 1 includes an aspiration catheter 2, a vacuum tube 4 for receiving tissue or fluid (e.g., a clot, blood, and so forth) from a patient (not shown) during a thrombectomy procedure. The tissue or fluid travels from the aspiration catheter 2 through the vacuum tube 4 to a collection container 6. Suction is provided through the vacuum tube 4 via a pump 8 (e.g., an aspiration pump) that includes an on/off switch 9. The apparatus 1 can also include a booster syringe 5 connected to the vacuum tube 4 via a T-connector 7. Each of these components of the apparatus 1 can be made from a clear polymer resin material to allow a user to visualize a clot of blood within the apparatus 1.

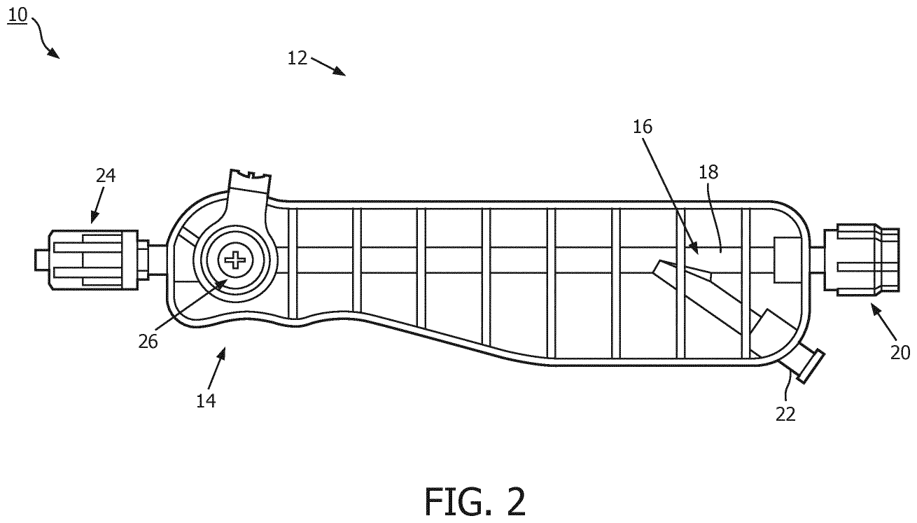

The thrombectomy device 1 also includes a thrombectomy device 10 having a hemostasis valve Y-connector 12. Referring now to FIG. 2 showing an enlarged view of the hemostasis valve Y-connector 12 in isolation, and with continuing reference to FIG. 1, the hemostasis valve Y-connector 12 includes a distal end 14 configured to rotatably connect with the aspiration catheter 2, and a Y-connector 16. The Y-connector 16 includes a first branch 18 connecting the distal end 14 with a hemostasis valve 20, and a second branch 22 connected to the vacuum tube 4. In some embodiments, the distal end 14 of the hemostasis valve Y connector 12 includes a catheter rotation control 24 operable to rotate the aspiration catheter 2 (shown only in FIG. 1) relative to the hemostasis valve Y connector 12.

The thrombectomy device 10 also includes an adjustable valve 26. In the illustrated embodiment of FIGS. 1 and 2, the adjustable valve 26 is integrated into the hemostasis valve Y connector 12 (e.g., at the distal end 16). In other embodiments, the adjustable valve 26 is configured to connect in-line with the vacuum tube 4. The adjustable valve 26 is configured to adjust a flow rate of fluid drawn by the aspiration pump 8 and having a closed position with zero flow rate, a maximum open position with a maximum flow rate, and at least one intermediate position with a flow rate intermediate between zero flow rate and the maximum flow rate. In some embodiments, the at least one intermediate position comprises a continuum of intermediate positions continuously spanning from zero flow rate to the maximum flow rate.

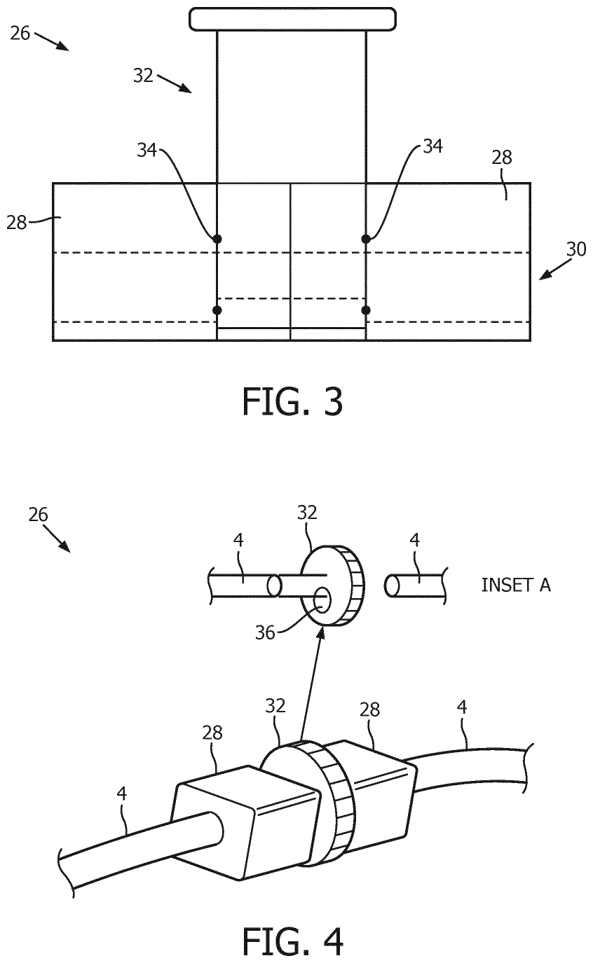

FIGS. 3 and 4 show examples of the adjustable valve 26. To adjust the fluid flow rate, the adjustable valve 26 includes a housing 28 defining a valve lumen 30, and a control mechanism 32 configured to adjustably occlude the valve lumen 30 to adjust the flow rate of fluid drawn by the aspiration pump 8. Advantageously, the adjustable valve 26 does not adjust the flow rate of fluid drawn by the aspiration pump 8 by compressing a tube defining the valve lumen 30. Compression of the vacuum tube 4 provides less reproducible flow rate control, and can result in weakening over time and eventually rupture of the tube in the area of compression.

FIG. 3 shows an example of the control mechanism 32 as a push-button (or plunger) 32 configured to adjustably occlude the valve lumen 30 to adjust the flow rate of fluid drawn by the aspiration pump 8. An operator can use, for example, his or her thumb to press the push-button 32 downward to control a size of a diameter of the valve lumen 30. In some examples, the push-button 32 can be secured to the housing 28 via one or more O-rings 34.

FIG. 4 shows an example of the control mechanism 32 as a rotary disk 32 configured to adjustably occlude the valve lumen 30 to adjust the flow rate of fluid drawn by the aspiration pump 8. As shown in FIG. 4, the rotary disk 32 includes an opening 36 having a diameter that substantially matches a diameter of the vacuum tube 4 (as shown in INSET A of FIG. 4). An operator can use, for example, his or her thumb to rotate the rotary disk 32 to control offset of the opening 36 respective to the valve lumen 30 in order to partially or entirely occlude the valve lumen 30 and thereby provide continuously adjustable flow control between zero flow rate (when the opening 36 is offset so far the lumen is entirely blocked) and maximum flow rate (when the opening 36 is exactly aligned with the valve lumen 30). In some variant embodiments, the rotary disk 32 can include a plurality of different-sized openings 36 providing stepwise-adjustable occlusion of the valve lumen 30.

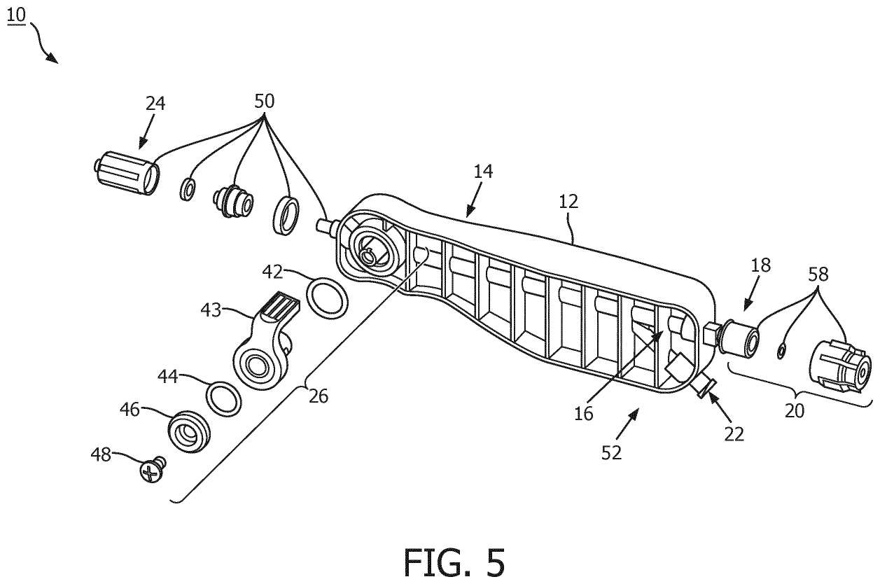

FIGS. 5-7 show a partially exploded perspective view of another embodiment of the thrombectomy device 10. As shown in FIG. 5, the catheter rotation control 24 comprises a flow control lever 24 that has the adjustable valve 26 integrated thereto. The flow control lever 24/adjustable valve 26 piece is secured to the distal end 14 of the hemostasis valve Y connector 12 via a large O-ring 42, a small O-ring 44, an end cap 46, and a screw 48. FIG. 5 also shows a rotating male luer connector 50 disposed at the distal end 14 of the hemostasis valve Y connector 12. An opposing proximal end 52 of the hemostasis valve Y connector 12 includes the Y-connector 16 the first branch 18 terminating in the hemostasis valve 20 that includes a compression cap with seal 58 and the second branch 22 terminating an aspiration port 60. The aspiration port 60 can comprise a luer fitting that can couple to the vacuum tube 4.

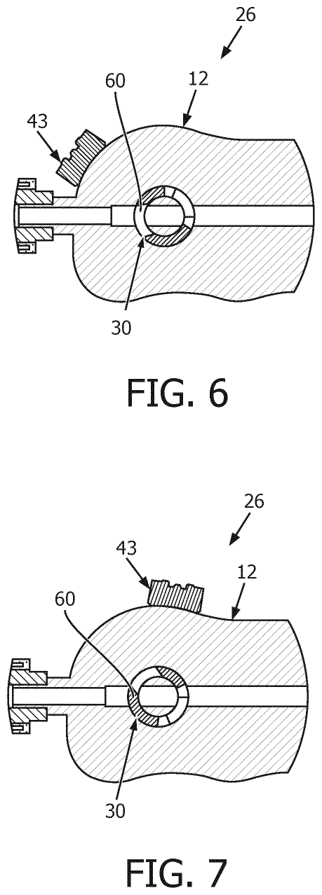

FIGS. 6 and 7 show operation of the flow control valve 26 by way of side sectional views. When the flow control lever 43 is in a “forward” position (as shown in FIG. 6, in which the flow control lever 43 is disposed towards the vacuum tube 4), an occlusion ring 60 connected with the flow control lever 43 is moved to a “full open” state, in which the occlusion ring 60 does not block the valve lumen 30 and hence suction can flow from the pump 8 through the vacuum line 4 and to the treatment site and allow suction of a clot. On the other hand, FIG. 7 shows the flow control lever 43 in a “rear” position (i.e., on top of the hemostasis valve Y connector 12 away from the vacuum line 4). As shown in FIG. 7, the adjustable valve 26 is then in a “closed” state in which the occlusion ring 60 is rotated to fully occlude the valve lumen 30, thereby preventing suction through the vacuum line 4. To move between the forward and rear positions, a user can use his or her thumb to control forward motion of the flow control lever 43. Moreover, at positions of the flow control lever 43 that are intermediate between the “full open” position of FIG. 6 and the “fully closed” position of FIG. 7, the flow rate is at an intermediate flow rate between the full open flow rate of FIG. 6 and the zero flow rate of FIG. 7, due to the occlusion ring 60 partially occluding the valve lumen 30. The flow rate is thus continuously adjustable between closed and full open by moving the flow control lever 43 gradually forward.

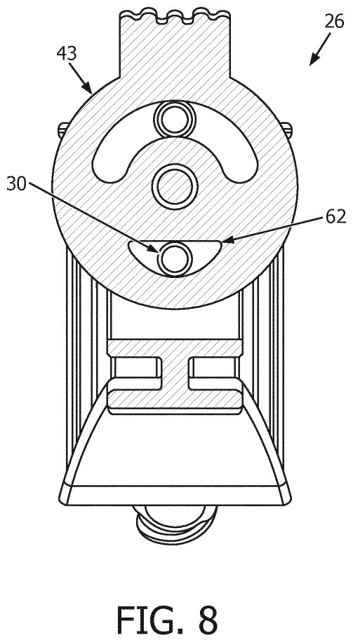

FIG. 8 shows another embodiment of the adjustable valve 26, which employs a flow control lever 43 but with a different design for occluding the flow. The flow control lever 43 has a wedge-shaped opening 62 (more generally, flow control feature 62) has the shape of a crescent moon that aligns with a valve lumen 30 to permit more gradual control of the flow rate between zero flow rate and full open flow rate. Notably, because the wedge-shaped opening 62 tapers as it approaches the fully closed position, it provides more precise control in the low flow rate range, which is useful when the operator is drawing blood while moving the aspiration catheter tip into engagement with clot material.

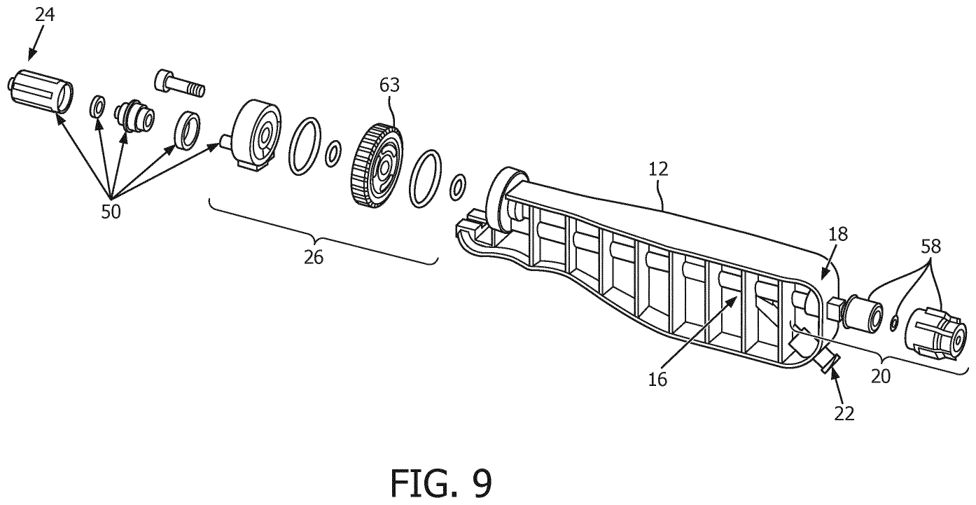

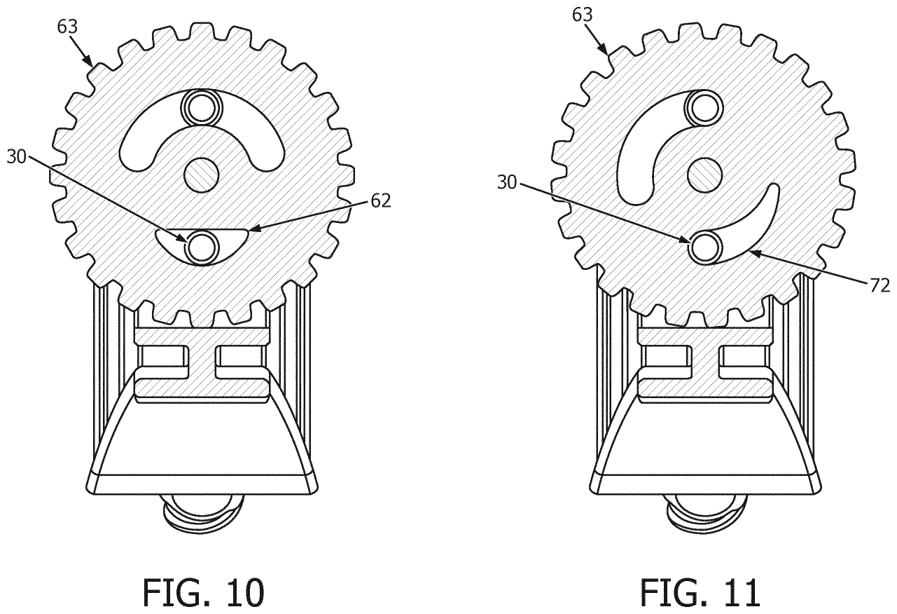

FIGS. 9-12 show some further illustrative embodiments. FIG. 9 shows an arrangement which is similar to the embodiment of FIG. 5, e.g., including the catheter rotation knob 24 and its internal mechanism 50 shown in exploded view at one end of the hemostasis valve Y-connector 12, and the Y-connector 16 with the hemostasis valve 20 at the other end, with the other branch of the Y-connector 16 providing connection to the aspiration port 22. However, as opposed to the forward motion of the flow control lever 43 shown in FIGS. 5-7, in the embodiment of FIG. 9 a flow control dial 63 can be moved from side-to-side to adjust the flow rate through the adjustable valve 26. FIGS. 10-12 illustrate three embodiments of the flow control dial 63, each with different flow control features.

The embodiment of FIG. 10 has the same crescent moon wedge shaped opening 62 as the flow control feature as in FIG. 8, and operates analogously to the operation of the example of FIG. 8 except that the dial 63 serves as the manual control rather than the flow control lever 43 as in the embodiment of FIG. 8.

FIG. 11 shows an example of another wedge-shaped opening 72 as the flow control feature. The wedge-shaped opening 72 is asymmetric and provides a larger rotation of the dial 63 to move between fully closed and fully open, thus providing finer control of the flow rate. Notably, both the embodiment of FIG. 10 and the embodiment of FIG. 11 provide continuous flow control, that is, there is a continuum of intermediate positions with flow rates that increase from zero flow rate to the maximum flow rate.

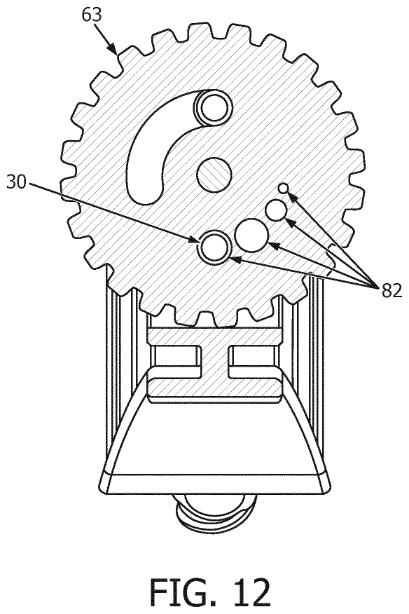

Unlike the embodiments of FIGS. 10 and 11, the embodiment of FIG. 12 provides a discrete number of intermediate flow rates flow rate intermediate between zero flow rate and the maximum flow rate. Specifically, the dial 63 of FIG. 12 includes four separate and distinct openings 82 that can be successively aligned with the valve lumen 30 as the dial 63 is rotated. This provides 5 positions: a fully closed position (none of the holes aligned with the valve lumen 30); three successively higher intermediate flow rate positions corresponding to openings of successively larger diameter being aligned with the valve lumen 30; and a fully open position corresponding to the largest-diameter opening being aligned with the valve lumen 30 (this latter full-open position being specifically shown in FIG. 12). In some embodiments, the dial 63 may include detents (not shown) or the like to lock movement of the dial 30 into the five discrete positions.

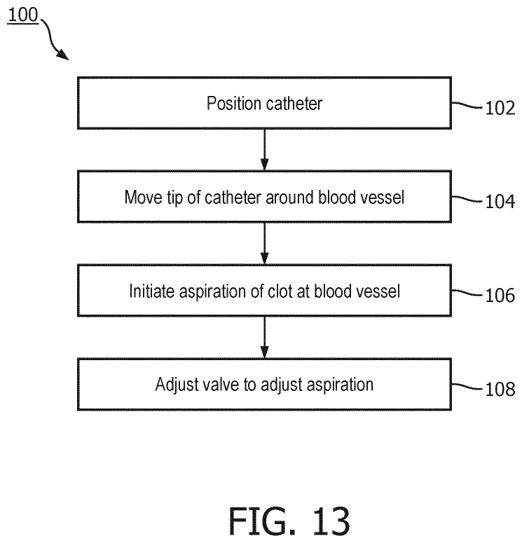

The thrombectomy device 10 can be used to perform a thrombectomy method 100 on a patient. Referring to FIG. 13, and with continuing reference to FIGS. 1-4, an illustrative embodiment of the thrombectomy method 100 is diagrammatically shown as a flowchart. At an operation 102, the catheter 2 is positioned adjacent a treatment site (e.g., a clot) of the patient. At an operation 104, the hemostasis valve Y connector 12 connected to the catheter 2 is operated by a user to move a tip of the catheter 2 radially around a blood vessel at the treatment site. At an operation 106, the adjustable valve 26 is operated to initiate aspiration of material (e.g., the clot) via the catheter 2 via suction provided by the pump 8 through the vacuum tube 4. At an operation 108, the adjustable valve 26 is adjusted to adjust the aspiration to a low but not zero flow rate of aspirated material when aspirated material transitions from clot material to blood.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A thrombectomy device, comprising:
a hemostasis valve Y connector including:
a distal end configured to rotatably connect with an associated aspiration catheter; and
a Y connector connecting the distal end with a first branch terminating in a hemostasis valve and a second branch configured to connect with an associated vacuum tube of an associated aspiration pump; and
an adjustable valve integrated into the hemostasis valve Y connector or configured to connect in-line with the associated vacuum tube, the adjustable valve configured to adjust a flow rate of fluid drawn by the associated aspiration pump and having a closed position with zero flow rate, a maximum open position with a maximum flow rate, and at least one intermediate position with a flow rate intermediate between zero flow rate and the maximum flow rate.

2. The thrombectomy device of claim 1, wherein the adjustable valve comprises:
a valve lumen; and
a control mechanism configured to adjustably occlude the valve lumen to adjust the flow rate of fluid drawn by the associated aspiration pump.

3. The thrombectomy device of claim 2, wherein the control mechanism comprises a push-button configured to adjustably occlude the valve lumen to adjust the flow rate of fluid drawn by the associated aspiration pump.

4. The thrombectomy device of claim 2, wherein the control mechanism comprises a rotary disk configured to adjustably occlude the valve lumen to adjust the flow rate of fluid drawn by the associated aspiration pump.

5. The thrombectomy device of claim 4, wherein the rotary disk comprises a plurality of different-sized openings providing the adjustable occlusion of the valve lumen.

6. The thrombectomy device of claim 2, wherein the adjustable valve does not adjust the flow rate of fluid drawn by the associated aspiration pump by compressing a tube defining the valve lumen.

7. The thrombectomy device of claim 1, wherein the adjustable valve is integrated into the hemostasis valve Y connector.

8. The thrombectomy device of claim 7, wherein:
the distal end of the hemostasis valve Y connector includes a catheter rotation control operable to rotate the associated aspiration catheter relative to the hemostasis valve Y connector; and
the adjustable valve is integrated into the distal end of the hemostasis valve Y connector.

9. The thrombectomy device of claim 1, wherein the adjustable valve is configured to connect in-line with the associated vacuum tube.

10. The thrombectomy device of claim 1, further comprising:
said aspiration pump.

11. A thrombectomy device, comprising:
a hemostasis valve Y connector including:
a distal end configured to rotatably connect with an associated aspiration catheter; and
a Y connector connecting the distal end with a first branch terminating in a hemostasis valve and a second branch configured to connect with an associated vacuum tube of an associated aspiration pump; and
an adjustable valve integrated into the hemostasis valve Y connector or configured to connect in-line with the associated vacuum tube, wherein the adjustable valve comprises:
a valve lumen; and
a control mechanism configured to adjustably occlude the valve lumen to adjust the flow rate of fluid drawn by the associated aspiration pump;
wherein the adjustable valve is configured to adjust a flow rate of fluid drawn by the associated aspiration pump and having a closed position with zero flow rate, a maximum open position with a maximum flow rate, and at least one intermediate position with a flow rate intermediate between zero flow rate and the maximum flow rate.

12. The thrombectomy device of claim 11, wherein the control mechanism comprises a push-button configured to adjustably occlude the valve lumen to adjust the flow rate of fluid drawn by the associated aspiration pump.

13. The thrombectomy device of claim 11, wherein the control mechanism comprises a rotary disk configured to adjustably occlude the valve lumen to adjust the flow rate of fluid drawn by the associated aspiration pump.

14. The thrombectomy device of claim 13, wherein the rotary disk comprises a plurality of different-sized openings providing the adjustable occlusion of the valve lumen.

15. The thrombectomy device of claim 11, wherein the adjustable valve does not adjust the flow rate of fluid drawn by the associated aspiration pump by compressing a tube defining the valve lumen.

16. The thrombectomy device of claim 11, wherein the adjustable valve is integrated into the hemostasis valve Y connector.

17. The thrombectomy device of claim 16, wherein:
the distal end of the hemostasis valve Y connector includes a catheter rotation control operable to rotate the associated aspiration catheter relative to the hemostasis valve Y connector; and
the adjustable valve is integrated into the distal end of the hemostasis valve Y connector.

18. The thrombectomy device of claim 11, wherein the adjustable valve is configured to connect in-line with the associated vacuum tube.

19. The thrombectomy device of claim 11, further comprising:
said aspiration pump.

20. A thrombectomy method, comprising:
positioning a catheter adjacent a treatment site of a patient;
operating a hemostasis valve Y connector connected to the catheter to move a tip of the catheter radially around a blood vessel at the treatment site;
operating an adjustable valve integrated into the hemostasis valve Y connector or configured to connect in-line with an associated vacuum tube connecting an aspiration pump with the hemostasis valve Y connector to initiate aspiration of material via the catheter; and
adjusting the adjustable valve to adjust the aspiration to a low but not zero flow rate of aspirated material when aspirated material transitions from clot material to blood.
